(19) United States
(12) Patent Application Publication
(10) Pub. No.: US 2013/0046328 A1
(43) Pub. Date: Feb. 21, 2013

(54) Hemorrhoid Pressure Relief Device

(71) Applicant: David Bruce Bourque, Pasadena, TX (US)

(72) Inventor: David Bruce Bourque, Pasadena, TX (US)

(21) Appl. No.: 13/211,307

(22) Filed: Aug. 17, 2011

(51) Int. Cl. A61M 29/00

(52) U.S. Cl. 606/197

(57) ABSTRACT

A hemorrhoid pressure relief device having fluid fill inside a tube made of a supple, soft, and yet highly tensile strong material produces a gentle support and counter pressure over the hemorrhoid bulge resulting in a lowering of the differential pressure across the tissue wall of the hemorrhoid, instantly reducing pain. Due to the fluidity of the liquid fill the device conforms to the contour of the hemorrohidal bulge and the adjacent buttocks cheeks, while being held in place, naturally keeping the buttocks cheeks from rubbing against the homorrhoidal bulge. Owing to the cooling capacity of the fluid fill, the device provides further pain relief. The device is simple and easy to use. Due to its simplicity, the manufacturing cost is inexpensive and can be afforded by most people. The device is disposable and thus is sanitary.

### BACKGROUND OF THE INVENTION

1. Field of the Invention

The present invention relates to hemorrhoid pressure relief devices, and more particularly to hemorrhoid pressure relief devices that contain fluid inside a supple, smooth, yet highly tensile tube. The devices alleviate users' pain by eliminating adjacent tissue rubbing, giving an immediate cooling effect, and providing a gentle support and counter pressure over the hemorrhoid bulge.

2. Description of Related Art

Hemorrhoids are clusters of vascular tissue, smooth muscle, and connective tissue lined by the normal epithelium of the anal canal. In their physiological state they act as a cushion that aid the passage of stool. They become pathological or piles when swollen or inflamed after straining to pass a stool or due to the pressure of the enlarged uterus during pregnancy. In general, hemorrhoids are categorized into internal and external hemorrhoids. These categories are anatomically separated by the dentate (pectinate) line. Internal hemorrhoids originate above the pectinate line; external hemorrhoids lie below the pectinate line. External hemorrhoids are present with pain in the area surrounding the anus. They can be felt when they swell and may cause itching or pain with a bowel movement, as well as bleeding. Internal hemorrhoids are located in the inside lining of the rectum and cannot be felt. Most internal hemorrhoids are present with painless rectal bleeding. But large hemorrhoids that bulge from the anus may become painful if they swell and are squeezed by the muscles that control the anus. The condition becomes significantly symptomatic when the pathological hemorrhoidal vascular tissues start to prolapse from its normal position (above the anal sphincter) and protrude outside the anus. Pathological hemorrhoids (diseases) are very common and are estimated to occur in up to one-half of the population by age 50. It is also estimated that more than 50 million Americans suffered from hemorrhoids. This group of population includes middle aged to older people and even young pregnant women.

The use of belts or straps or other members for holding hemorrhoid is known in the field. For example, U.S. Pat. No. 4,702,237 to Gianopoulos et al. discloses a flat member which can be secured to the anal area to physically hold the hemorrhoids within the anal orifice. U.S. Pat. No. 6,712,841 to Gomez (2004) discloses materials to separate swollen, inflamed tissue from non-swollen tissue. The material incorporated into the undergarment to separate the buttocks. These devices can be uncomfortable and may be unsanitary if not disposable. Besides holding the hemorrhoid bulge in place or separating hemorrhoid from non-swollen tissue, neither of the devices provides additional pain relieving features.

Other devices are designed to push the protruding hemorrhoids back to its normal position inside anal canal. For instance, U.S. Pat. No. 6,364,852 B1 to Lee (2002) discloses a cylindrical device of about 5-30 mm in diameter. The device is inserted into an anal canal so that it is positioned against at least one displaced anal cushion, and is maintained there for a sufficient time to achieve reduction of the displaced anal cushion. The device is highly invasive; it requires insertion through an often painfully-inflamed anus; it could be messy and not sanitary. The device staying inside anal canal may affect users' functionality. due to uncomfortable sensation. Besides applying pressure to push hemorrhoids back, the device does not provide other pain relieving mechanisms. Likewise, U.S. Pat. Nos. 5,924,423 to Majlessi, 4,583,542 to Boyd, 5,178,627 to Hudock, 4,263,914 to Pawlak, and 3,712,300 to Davidowitz and 2007/0255307 A1 to Cummings, III (2007) share the same shortcomings of the aforementioned U.S. Pat. No. 6,364,852 B1 to Lee (2002). These known devices and apparatuses have thus been less than desirable for those desiring a sanitary and minimally invasive solution to the problem of hemorrhoids.

U.S. Pat. No. 6,716,229 B2 to Toth (2004) discloses a hemorrhoid relief and anal hygiene device comprising a pressure member (a bulbous central portion) for placement between opposing buttock surfaces adjacent to the anus of the user; and a pair of elongate side members coupled to and extending outwardly from opposed sides of the central portion. The device is constructed entirely of an absorbent, flexible, resilient material such as paper or cotton which may cause irritation to hemorrhoids because they don't have smooth surface. Besides providing counter pressure, the device does not offer other pain relief features such as cooling effect. Moreover, the shape and bulkiness of the device may affect users' functionality.

Therefore, the need still remains for a simple, easy to use, disposable, sanitary, inexpensive device that can alleviate hemorrhoid pain through multiple pain relief mechanisms.

The hemorrhoid pressure relief devices according to the present invention substantially overcome the shortcomings encountered in the aforementioned patents.

### SUMMARY OF THE INVENTION

The primary object of the present invention is to provide a device that can relieve pain caused by hemorrhoids.

Another object of the present invention is to provide a device that can promote healing of hemorrhoids.

Another object of the present invention is to provide a device that can prevent hemorrhoids from happening.

Yet another object of the present invention is to provide a device that is easy to use.

Still another object of the present invention is to provide a device that is disposable for hygiene concerns.

Finally, the present invention should also provide a device that is simple and cost effective to manufacture so that it is more affordable.

The device of the present invention is a tube made of materials that are supple, smooth, yet strong and has high tensile property including but not limited to vinyl or latex or nitrile or any combination thereof. The material suitable for the intended purpose should be capable of containing a fluid including but not limited to a liquid, a gel, and/or a gas. For example, the vinyl surgical glove material, which can maintain stretching and hold its volume even with more than 5 psi of pressure applied to it, is a preferable material.

The device may be in different shapes, preferably in a substantially cylindrical shape with two round or semi-round shaped ends. The device is about 1.5-3 inches, preferably about 2 inches in length, and about 0.5-1 inch, preferably about 0.75 inch in diameter. The device has a wall of approximately the same thickness as a typical surgical glove which may be about 0.15-0.3 mm, preferably 0.25 mm. The device is slightly under filled with fluid including but not limited to liquid, gel, and/or gas so that when it is placed over the hemorrhoid bulge, it can conform to the contour of the protrusion while keeping the buttocks cheeks from rubbing on the hemorrhoid tissue; thus it can alleviate pain. Preferably, the device is filled up to about 80%-99% of its fullness to achieve best pain relieving effect. The device may be kept in the refrigerator; after the device is removed from the refrigerator and immediately applied, the liquid or gel contained inside the device further reduces pain through cooling effect. The prototype of the device is constructed using one finger of a vinyl surgical glove and is made in a shape of a short tube, wherein water is filled up to about 80%-99% of its fullness.

The device of the present invention provides pain relief through three mechanisms. First, the device provides a gentle support and counter pressure over the external wall of the bulge resulting in a lowering of the differential pressure across the tissue wall of the hemorrhoid. The liquid filled inside the device rolls with the movement of the body eliminating movement over the bulge keeping the hemorrhoid immobile during activity. Secondly, the device keeps the buttocks cheeks from rubbing against the hemorrhoid bulge, reducing irritation to the hemorrhoids. Thirdly, due to the cooling capacity of the fluid fill, the device also cools the external hemorrhoid. For greater cooling, it can be placed in a refrigerator prior to use resulting in an enhanced cooling/soothing effect.

The device of the present invention not only mitigates pain of external hemorrhoids, it promotes healing of hemorrhoids by providing counter pressure to cancel off or reduce the pressure exerted on the bulging hemorrhoid tissue by internal body fluid and aid in pushing the external bulge back inside. It can also be used to prevent hemorrhoid tissues from protruding out of anal canal.

The device is simple and easy to use. The user takes the device from the package and places it directly onto the hemorrhoid bulge while in a sitting position, preferably sitting on a toilet. It will be naturally held (pinched) in place by the buttock. The device starts to work immediately by conforming to and supporting the exterior wall of the distended tissue. The device provides support to the hemorrhoid while the user is standing or walking. However, the best pain soothing effect is achieved when the user sits down because while sitting buttocks squeezes the device and the internal pressure of the device increases which in turn applies more counter pressure against the hemorrhoid bulge. The natural pressure placed upon the device by sitting down enhances the therapeutic effect. The device may further comprise fastening means on one side for attachment to the undergarment or buttocks. The device may have lubricant on one side that is in contact with hemorrhoid bulge.

The device can be used in combination with other hemorrhoids treatment including but not limited to medications such as Preparation-H®.or a Cooling Jel The device can be used as a stand-alone product in conjunction with other hemorrhoids medications for treatment or the device can further comprise hemorrhoids medication on one side of the device for applications onto the hemorrhoids for benefits of both devices and medications.

Due to its simplicity and easy to manufacture, the cost to construct a working device according to the present invention is probably a couple of pennies each. It is so inexpensive to manufacture that it can, and should be, disposable for hygiene concerns; the device is affordable to most people.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a perspective side view of an embodiment of the present invention;

FIG. 2 is a perspective view showing the placement position of the device of the invention in a sitting position when the device is first placed over the hemorrhoid bulge;

FIG. 3 is a perspective view showing the position of the device of the invention while standing or sitting on a chair; and

### DESCRIPTION OF THE PREFERRED EMBODIMENT

Referring to FIG. 1, there is disclosed a perspective side view of an embodiment of the device according to the present invention. The device 10 is a short tube 101 made of a soft (supple) yet strong and highly tensile material capable of holding fluid 102 including but not limited to liquid or gas inside under pressure. The device 10 is in a cylindrical shape having about 2 inches in length, about 0.75 inch in diameter. The wall of the short tube 101 is approximately as thick as a typical surgical glove which may be about 0.15-0.3 mm. The device 10 is slightly under filled so that when it is placed over the bulge, it can conform to the contour of the protrusion 20 while keeping the buttocks cheeks 30 from rubbing on the hemorrhoid tissue.

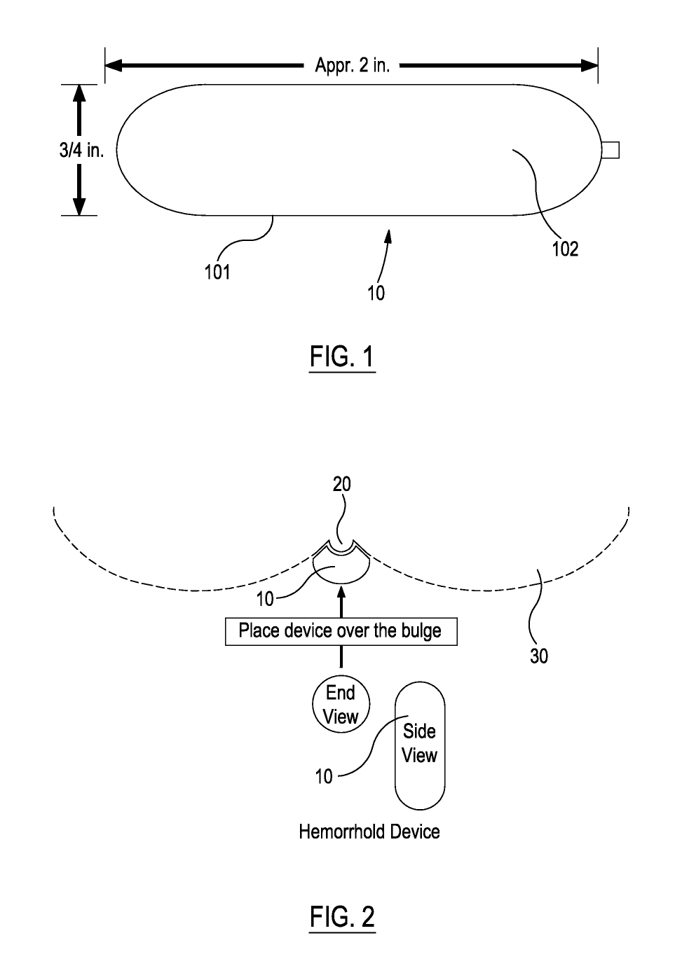

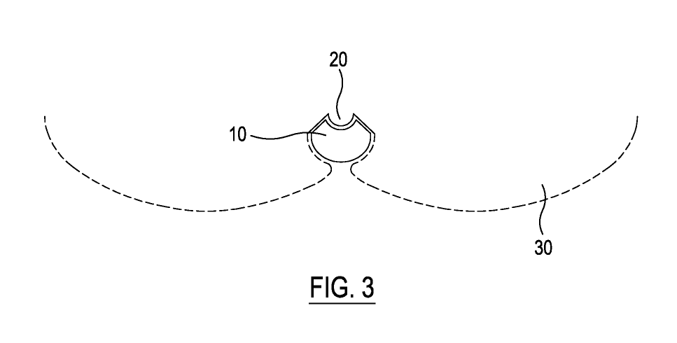

FIG. 2 shows a perspective view of the position of the device 10 when it is first placed over the hemorrhoid bulge 20 between buttocks cheeks 30 in a sitting position, preferably sitting on a toilet. FIG. 3 shows a perspective view of the position of the device 10 after the device 10 is placed over the hemorrhoid bulge 20, the device 10 is then held in place by the natural contour of the body while standing or sitting. Due to flexibility the device 10 conforms to the contour of the bulge 20 so it acts as a zero friction cushion between the hemorrhoids bulge 20 and the buttocks 30, and thus significantly reduces the pain coming from the rubbing on the hemorrhoids bulge 20 by buttocks cheeks 30. The size and shape of device 10 may be reduced/changed under pressure from hemorrhoid bulge 20 and the buttocks 30 as shown in FIG. 2. Moreover, the fluid fill 102 inside the tube 101 presses against the hemorrhoid bulge 20 reducing the pressure caused by the internal body fluid onto the hemorrhoid bulge 20, and thus greatly easing the pain. The device works best in a sitting position because sitting places pressure on the hydraulics of the device 10 causing the bulge 20 to move back through the anus where it no longer causes pain. Nevertheless, the device also works while standing or walking by the same reasons described above although the effect is not as great as in a sitting position.

One of the embodiments further comprises a medical strip on one side of its wall for treating hemorrhoid. In use, the side with medication strip is placed in contact with the hemorrhoid bulge so that the user can benefit from both medication and device.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A hemorrhoid pressure relief device comprises:
a short tube; and a fluid within the tube; the tube is made of supple, smooth, yet highly tensile strong material, the tube is capable of containing a fluid under pressure, the device being placed over hemorrhoid bulge between opposing buttock cheeks having sufficient flexibility so as to conform to and held in place by user's natural contour, the device provides a gentle and constant pressure onto the external hemorrhoid wall diminishing pain due to tissue distention.

2. The hemorrhoid pressure relief device of claim 1, wherein the short tube being substantially cylindrical in shape of between 1.5 and 3 inches in length and between 0.5 and 1 inch in diameter.

3. The hemorrhoid pressure relief device of claim 1, wherein the short tube may be in any shapes.

4. The hemorrhoid pressure relief device of claim 1, wherein the short tube has a wall of approximately the same thickness as a typical surgical glove.

5. The hemorrhoid pressure relief device of claim 4, wherein the wall of the short tube is between 0.15 mm and 0.30 mm.

6. The hemorrhoid pressure relief device of claim 1, wherein the material of the tube is vinyl.

7. The hemorrhoid pressure relief device of claim 1, wherein when filled to full capacity of 100% acts as a cap or preventive measure to keep the hemorrhoid from pushing out.

8. The hemorrhoid pressure relief device of claim 1, wherein the device is used after a bowel movement where excessive straining results in prolapsed tissue.

9. The hemorrhoid pressure relief device of claim 1, wherein the fluid within the tube is one of liquid, gel, or gas.

10. The hemorrhoid pressure relief device of claim 9, wherein the fluid within the tube is water.

11. The hemorrhoid pressure relief device of claim 1 further comprising medication on one side for application on the hemorrhoid bulge.

12. The hemorrhoid pressure relief device of claim 1 further comprising fastening means on one side for attachment to the undergarment or buttocks.

13. The hemorrhoid pressure relief device in claim 1 further immobilizes the hemorrhoid bulge.

14. The hemorrhoid pressure relief device in claim 1 further separates the hemorrhoid from adjacent body tissue.

15. The hemorrhoid pressure relief device in claim 1 further helps to push the prolapsed internal hemorrhoid back inside to its original position.

16. The hemorrhoid pressure relief device in claim 1 is refrigerated prior to use providing a cooling relief to the hemorrhoid.

17. The hemorrhoid pressure relief device of claim 1 is simple and easy to use.

18. The hemorrhoid pressure relief device of claim 1 is disposable.

19. A hemorrhoid pressure relief device comprises:
a short tube made of vinyl material; and liquid filled within the tube; wherein the short tube being a cylinder of about 1.5 to 3 inches in length and about 0.5 to 1 inch in diameter and a wall of approximately the same thickness as a typical surgical glove, the device being placed over hemorrhoid bulge between buttock cheeks having sufficient flexibility so as to conform to and held in place by user's natural contour, the device provides a gentle and constant pressure onto the external hemorrhoid wall diminishing pain, the device further immobilizing the hemorrhoid bulge, separating the hemorrhoid from adjacent body tissue, providing a cooling relief to the hemorrhoid.

20. A hemorrhoid pressure relief device comprises:
a short tube made of vinyl material; and water filled within the tube; wherein the short tube being a cylinder with two round shaped ends of about 1.5 to 3 inches in length and about 0.5 to 1 inch in diameter and a wall of approximately the same thickness as a typical surgical glove, the device being placed over hemorrhoid bulge between buttock cheeks having sufficient flexibility so as to conform to and held in place by user's natural contour, the device provides a gentle and constant pressure onto the external hemorrhoid wall diminishing pain, the device further immobilizing the hemorrhoid bulge, separating the hemorrhoid from adjacent body tissue, providing a cooling relief to the hemorrhoid.
